(19) United States
(12) Patent Application Publication
(10) Pub. No.: US 2024/0016871 A1
(43) Pub. Date: Jan. 18, 2024

(54) Non-toxic furocoumarin-rich plant extracts and related compositions for use as antimicrobial substance or medicament and other therapeutic uses

(71) Applicant: ICE-P LIFE, S.L., Barcelona (ES)

(72) Inventor: Ezio PANZERI, Alicante (ES)

(21) Appl. No.: 18/251,158

(22) PCT Filed: Aug. 28, 2021
(86) PCT No.: PCT/IB2021/057686

§ 371 Date: Apr. 28, 2023

(30) Foreign Application Priority Data
Oct. 28, 2020 (GB) 2017123.7

(51) Int. Cl. A61K 36/232; A61P 31/00

(52) U.S. Cl. CPC A61K 36/232; A61P 31/00

(57) ABSTRACT

The present invention refers to a furocoumarin-rich extract for use as antimicrobial substance, drug or medicament which comprises an extract of Angelica archangelica. The invention also refers to a non-toxic Angelica plant species furocoumarin-rich extract produced by a described method and to a said method of producing the extract. The Angelica archangelica furocoumarin-rich extract produced as described has shown a marked significant in vitro cytoprotective effect in SARS-CoV-2-exposed Vero E6 cellular cultures. The non-toxic furocoumarin-rich extract may be used for treating pathogenic infections of nucleic acid-regulated microbiome including but not restricted to virus, bacteria, parasites and fungus affecting animals and human beings. In particular, it may be used for the treatment of viral infections such as HIV infections, Dengue, Influenza, Hepatitis B, Hepatitis C, measles, mumps, herpes simplex, poliovirus, canine hepatitis, retrovirus, similar lentivirus, and in the treatment of current and future coronavirus infections, such as COVID-19, among others. Additionally, the non-toxic furocoumarin-rich extracts may be used the treatment of cancer of different aetiologies.

### CROSS-REFERENCE TO RELATED APPLICATION

This application is a National Stage Application of PCT Application No. PCT/162021/057686, filed on Aug. 20, 2021, which claims the benefit of and priority to United Kingdom Patent Application No. 2017123.7, filed on Oct. 28, 2020, titled NON-TOXIC FUROCOUMARIN-RICH PLANT EXTRACTS AND RELATED COMPOSITIONS FOR USE AS ANTIMICROBIAL SUBSTANCE OR MEDICAMENT AND OTHER THERAPEUTIC USES the entire contents of which are hereby incorporated by reference as if set forth in their entirety.

### TECHNICAL FIELD

The present invention belongs to the technical field of plant extracts and related active principles for their use as antimicrobial substances, herbal drugs or medicaments. In particular, the invention refers to non-toxic furocoumarin-rich extracts and their furocoumarin molecules for their use as antimicrobial substance, herbal drug or medicament. The provided non-toxic furocoumarin-rich extracts and related compounds and compositions are suitable for treating pathogenic infections of nucleic acid-regulated microbiome including but not restricted to virus, bacteria, parasites and fungus affecting animals and human beings. More particularly, the invention refers to non-toxic furocoumarin-rich extracts and related compositions of the Angelica plants family for their use in the treatment of severe acute respiratory syndrome coronavirus 2 (SARS-CoV-2), aetiology of coronavirus disease 2019 (COVID19), and potential COVID19-related future viral linages or strains.

### BACKGROUND OF THE INVENTION

Coumarins comprise a large class of compounds found throughout the plant kingdom [1-3]. They are found at high levels in some essential oils, particularly cinnamon bark oil (7,000 ppm), cassia leaf oil (up to 87,300 ppm) and lavender oil. Coumarin is also found in fruits (e.g. bilberry, cloudberry), green tea and other foods such as chicory [4]. Most coumarins occur in higher plants, with the richest sources being the Rutaceae and Umbelliferae. Although distributed throughout all parts of the plant, the coumarins occur at the highest levels in the fruits, followed by the roots, stems and leaves. Environmental conditions and seasonal changes can influence the occurrence in diverse parts of the plant [5].

Toxicology

Since 1954, coumarin has been classified as a toxic substance by the FDA, following reports of its possible liver tumour-producing properties in rats [6]. The FDA banned its use, labelling as adulterated all foods containing coumarin [7]. Due to tests performed on rodents coumarin was referred to as a chemical carcinogen by NIOSH [National Institute for Occupational Safety and Health]. However, caution needs to be taken in extrapolating this information to human situations. Various tests (Ames, micronucleus) have shown that coumarin and its metabolites are non-mutagenic [1]. Preliminary results from early studies indicated that coumarin was a toxin, but it has been shown since, that the rat is a poor model to compare with the human for this metabolism [8]. Several studies have examined the acute, chronic and carcinogenic effects of coumarin in the rat and mouse. In studies involving the rat, hepatic biochemical and morphological changes have been examined for various periods of coumarin administration (1 week to 2 years). Depending on dose administered, coumarin treatment results in an increase in relative weight and changes in various hepatic biochemical parameters. Single oral doses of coumarin have been shown to produce liver necrosis and increase plasma transaminase activities in DBA/2 strain mice [4].

Psoralene is a compound of origin in a family of natural products known as furanocoumarins with formula C11H6OR3. It is structurally related to coumarin by the addition of a furan ring and can be considered as a derivative of umbeliferone. Psoralene is naturally produced in the seeds of Psoralea corylifolia, as well as in Ficus carica, celery, parsley and Zanthoxylum. It is widely used in UVA plus psoralene therapy (PUVA) in the treatment for psoriasis, eczema, vitiligo, and skin T-cell lymphoma. Many furocumarins are extremely toxic to fish, and some are deposited in streams in Indonesia to catch fish. Ficus carica (fig tree) is probably the most abundant source of psoralenes. They are also found in small amounts in Ammi visnaga, Pastinaca sativa, Petroselinum crispum, Levisticum officinale, Foeniculum vulgare, Daucus carota, Psoralea corylifolia and Apium graveolens.

Psoralene is a mutagen and is used for this purpose in molecular biology research. Psoralene is interspersed in DNA and when exposed to ultraviolet radiation (UVA) can form covalent monoaducts and intercatenary crosslinks (ICL) with timines, preferably at 5′-TpA sites in the genome, inducing apoptosis. UVA plus psoralene therapy (PUVA) can be used to treat hyperproliferative skin disorders such as psoriasis and certain types of skin cancer [9]. Unfortunately, PUVA treatment alone carries an increased risk of skin cancer [10].

An important use of psoralene is in the treatment with PUVA for skin problems such as psoriasis and (to a lesser extent) eczema and vitiligo. This takes advantage of the high UV absorbance of psoralene. Psoralene is first applied to sensitize the skin, then UVA light is applied to cleanse the skin problem. Psoralene has also been recommended to treat alopecia [11]. Psoralenes are also used in photopheresis, where they are mixed with the leukocytes extracted before UV radiation is applied.

Despite the photocarcinogenic properties of some psoralenes [12, 13], it was used as a tanning activator in sunscreens until 1996 [14]. Psoralenes are used in tanning accelerators, as psoralene increases skin sensitivity to light. Some patients have had severe skin loss after sunbathing with tanning activators containing psoralene [15]. Patients with a lighter skin colour suffer four times more from the melanoma-generating properties of psoralenes than those with darker skin [14]. Short-term side effects of psoralene include nausea, vomiting, erythema, itching, xerosis, skin pain due to phototoxic damage to the dermal nerve and can cause skin and genital malignancies of the skin [16].

An additional use for optimized psoralenes is for inactivation of pathogens in blood products. Synthetic amino-psoralene, amotosalen HCl, has been developed for the inactivation of infectious pathogens (bacteria, viruses, protozoa) in the blood components of platelets and plasma prepared for transfusion support in patients. Prior to clinical use, platelets treated with amotosalen have been tested and found to be non-carcinogenic (after filtration & removal of amotosalen conjugated with pathogens) when using the standard p53 knockout mouse model [17]. The technology is currently routinely used in certain European blood centers and has recently been approved in the US [18, 19, 20, 21]. An example of a haemoglobin conjugate with such compounds is described by the inventor Mr. Ezio Panzeri in U.S. Pat. No. 7,235,639 B2.

An herbal extract derived from the root of the plant Angelica sinensis with possible anti-inflammatory, antispasmodic, vasodilatory, estrogenic, and antitumor activities. Angelica sinensis contains volatile oils, including safrole, isosafrole, and n-butylphthalide; coumarin derivatives, including psoralens, bergapten, osthol, imperatorin, and oxypeucedanin; and ferulic acid. The coumarin derivatives in this agent may vasodilate and relax smooth muscle and may exhibit additive anticoagulant effects. Ferulic acid, a phenolic phytochemical present in plant cell walls, may neutralize free radicals such as reactive oxygen species. In addition, Angelica sinensis extract has been shown to inhibit the growth and induce apoptosis of glioblastoma multiforme brain tumor cells through p53-dependent and p53-independent pathways. [22].

Structure

The structure of psoralene was originally inferred by identifying the products of their degradation reactions. It exhibits normal reactions of coumarin lactone, such as opening the ring by alkali to give a cumarinic acid or a derivative of cumaric acid. Potassium permanganate causes the oxidation of the furan ring, while other oxidation methods produce furan-2,3-carboxylic acid.

Angelicine is a psoralene isomer and most furocumarins can be considered as derivatives of psoralene or angelicine. Important derivatives of psoralene include imperatorine, xanthoxin, bergaptene and nodakenetine.

Biosynthesis

Psoralene is biosynthesized from umbeliferone, a coumarin derived from the phenylpropanoid route, from the aromatic amino acid Tyrosine, on the path of shikimic acid.

Considering biosynthesis from coumarin, the route consists of the following stages:
a) Prenylation of the aromatic ring: Dimethylylethyl pyrophosphate (DMAPP) forms an electrophilic intermediary, which reacts with the aromatic ring activated in ortho by the phenolic hydroxyl of coumarin, in a reaction similar to the alkylation of Friedel-Crafts. The product is an o-isopentenil (called“prenil”) 21′ phenol. In the case of umbeliferone, the product has been identified as demethylsuberosine.
b) Oxidative heterocyclic endocyclation: The dissused vinyl carbon of the prenile substitute is oxidized with oxygen to form an alcohol, and its neighbouring vinyl position is activated as an electrophilic, where phenolic hydroxyl is added in such a way that a dihydrobenzofuranoid ring is formed. The product of this stage is called marmesin. This reaction is catalysed by a cytochrome P450 and NADH dependent monooxygenase as coenzyme.
c) Oxidative excision: The oxidative rupture with oxygen of the isopropyl substitute of the diffuse ring of marmesin produces acetone and an unsaturation that aromatizes the furanoid ring. At this stage the psoralene itself is formed. This stage is performed by a second cytochrome P450 dependent monooxygenase. It is postulated that the mechanism is radicalary.

Psoralens are natural products, linear furanocoumarins (most furanocoumarins can be regarded as derivatives of psoralen or angelicin), present in several plant families that are extremely toxic to a wide variety of prokaryotic and eukaryotic organism. They may react directly with pyrimidine nucleotides forming mono and di adducts in DNA of even interstrand cross links. Some important psoralen derivatives are Xanthotoxin, Imperatorin, Bergapten and Nodekenetin.

Another cause of their toxicity derives from the ability of UV-A photoactivated furanocoumarins to react with grand state oxygen generating toxic oxyradicals capable of inactivating proteins within cells.

Pharmaceutical Uses

This reactivity has suggested their use as pharmaceuticals for a broad range of therapeutics applications requiring cell division inhibitors, vitiligo, psoriasis and several type of cancers like T cell lymphoma; main drug targets is the cytochrome P450 (CYPs superfamily), the inactivation mechanism of P450 by psoralen is not completely understood, may be 3 ways: a) binding of the inhibitor to the apoprotein, b) binding of the inhibitor to the heme, c) reaction of the inhibitor with the heme inducing fragmentation, apoptosis can be triggered and programmed unless repaired by cellular mechanisms.

The genus Angelica Litoralis is comprised of over 90 species spread throughout most areas of the globe [23]. More than half of these species are used in traditional therapies, while some of them are included in several national and European pharmacopoeias. Bioactive constituents in different Angelica species include coumarins, essential oils, polysaccharides, organic acids and acetylenic compounds [24]. In vitro testing confirmed cytotoxic [25, 26], anti-inflammatory [27], antibacterial [28], antifungal [29], neuroprotective [30], serotonergic activities for extracts obtained from a range of Angelica species.

Although the chemical composition of the different species constituted the object of numerous studies so far, a survey of the literature reveals little data regarding physiological processes in these plants (though see 32). This holds true especially for less studied species such as Wild Angelica (A. sylvestris).

The antiviral remdesivir is the first drug to be licensed for the treatment of COVID-19 by the US FDA and has a conditional marketing license from its European counterpart. Remdesivir is being tested as a treatment for COVID19, and has been authorized for emergency use in India, Singapore, and approved for use in Japan, the European Union, the United States, and Australia for people with severe symptoms, due to its short supply. It is licensed for the treatment of COVID-19 in adults and adolescents (aged 12 years with body weight 0.40 kg) with pneumonia requiring supplemental oxygen.

Because remdesivir supplies are limited, and it is very difficult and expensive to produce, it is recommended prioritizing remdesivir for use in hospitalized patients with COVID-19 who require supplemental oxygen but who do not require oxygen delivery through a high-flow device, noninvasive ventilation, invasive mechanical ventilation, or extracorporeal membrane oxygenation.

Solidarity Therapeutics trials interim results indicated that the remdesivir, hydroxychloroquine, lopinavir/ritonavir and interferon drugs appeared to have little or no effect in reducing COVID19-confirmed 28-day mortality [33].

However, as already mentioned, the drug remdesivir is in very short supply and it is very difficult and expensive to produce and only a small proportion of COVID-19 infected population can have access to this antiviral treatment. According to international experts from the British Medical Journal, “the drug probably has no important effect on the need for mechanical ventilation and may have little or no effect on the length of hospital stay”. Because of the high price, the authors point out that remdesivir may divert funds and efforts away from other effective treatments against COVID-19 [34].

Therefore, there is a pressing need for additional, more widely available and more effective antiviral substances and/or medicaments to combat the current COVID-19 pandemic.

### REFERENCES

1) Egan D, O'Kennedy R, Moran E, Cox D, Prosser E, Thornes R D. The Pharmacology, Metabolism, Analysis and Applications of Coumarin and Coumarin-Related Compounds. Drug Metab Rev 1990; 22: 503-529.
2) Egan D, O'Kennedy R. Rapid and Sensitive Determination of Coumarin and 7-Hydroxycoumarin and its Glucuronide Conjugate in Urine and Plasma by High Performance Liquid Chromatography. J Chromatogr B 1992; 582: 137-143.
3) Finn G, Kenealy E, Creaven B, Egan D. In vitro Cytotoxic Potential and Mechanism of Action of Selected Coumarins, Using Human Renal Cell Lines. Cancer Letts 2002; 183: 61-68.
4) Lake B. Coumarin Metabolism, Toxicity and Carcinogenicity: Relevance for Human Risk Assessment. Food Chem Tox 1999; 37: 423-453.
5) Keating G, O'Kennedy R. The Chemistry and Occurrence of Coumarins. Coumarins: Biology, Applications and Mode of Action. (Eds: O'Kennedy R, Thornes R D), Chichester, John Wiley & Sons, 1997; pp 23-66.
6) Murray R D H, Mendez J, Brown S A. The Natural Coumarins—Occurrence, Chemistry and Biochemistry. Chichester, John Wiley, 1982.
7) Cooke D. Studies on the Mode of Action of Coumarins (Coumarin, 6-hydroxycoumarin, 7-hydroxycoumarin and Esculetin) at a Cellular Level. PhD Thesis, Dublin City University, Dublin, Ireland, 1999
8) Deasy B. Development of Novel Analytical Methods to Study the Metabolism of Coumarin. PhD Thesis, Dublin City University, Dublin, Ireland, 1996.
9) Wu Q, Christensen L A, Legerski R J, Vasquez K M (June 2005). “Mismatch repair participates in error-free processing of DNA interstrand crosslinks in human cells”. EMBO Rep. 6 (6): 551-7.
10) Momtaz K, Fitzpatrick T B (April 1998). “The benefits and risks of long-term PUVA photochemotherapy”. Dermatol Clin. 16 (2): 227-34.
11) “Alopecia Areata: Psoralen With Ultraviolet A Light (PUVA) Therapy-Topic Overview”
12) M. J. Ashwood-Smith; G. A. Poulton; M. Barker; M. Mildenberger E (1980). “5-Methoxypsoralen, an ingredient in several suntan preparations, has lethal, mutagenic and clastogenic properties”. Nature. 285 (5): 407-9.
13) Zajdela F, Bisagni E (1981). “5-Methoxypsoralen, the melanogenic additive in suntan preparations, is tumorigenic in mice exposed to 365 nm UV radiation”. Carcinogenesis. 2 (2): 121-7.
14) Autier P.; Dore J.-F.; Cesarini J.-P. (1997). “Should subjects who used psoralen suntan activators be screened for melanoma?”. Annals of Oncology. 8 (5): 435-7.
15) Nettelblad H, Vahlqvist C, Krysander L, Sjöberg F (December 1996). “Psoralens used for cosmetic sun tanning: an unusual cause of extensive burn injury”. Burns. 22(8): 633-5.
16) Shenoi, Shrutakirthi D.; Prabhu, Smitha; Indian Association of Dermatologists, Venereologists and Leprologists (November 2014). “Photochemotherapy (PUVA) in psoriasis and vitiligo”. Indian Journal of Dermatology, Venereology and Leprology. 80 (6): 497-504.
17) Ciaravino V, McCullough T, Dayan A D: Pharmacokinetic and toxicology assessment of INTERCEPT (S-59 and UVA treated) platelets. Human Exp Toxicol 2001; 20:533-550.
18) Osselaer; et al. (2009). “Universal adoption of pathogen inactivation of platelet components: impact on platelet and red blood cell component use”. Transfusion. 49(7): 1412-1422.
19) Cazenave; et al. (2010). “An active hemovigilance program characterizing the safety profile of 7,483 transfusions with plasma components prepared with amotosalen and UVA photochemical treatment”. Transfusion. 50 (6): 1210-1219.
20) https://www.fda.gov/NewsEvents/Newsroom/PressAnnouncements/ucm427111.htm
21) https://www.fda.gov/NewsEvents/Newsroom/PressAnnouncements/ucm427500.htm
22) “Angelica sinensis root extract”. NCI Thesaurus
23) Feng T, Downie S R, Yu Y, Zhang X, Chen W, He X, Liu S. Molecular systematics of Angelica and allied genera (Apiaceae) from the Hengduan Mountains of China based on nrDNA ITS sequences: phylogenetic affinities and biogeographic implications. J Plant Res 2009; 122(4):403-414.
24) Sarker S D, Nahar L. Natural Medicine: Genus Angelica. Curr Med Chem 2004; 11(11):1479-1500.
25) Lee, S., Lee, Y. S., Jung, S. H., Shin, K. H., Kim, B. K., Kang, S. S., 2003. Anti-Tumor Activities of Decursinol Angelate and Decursin from Angelica gigas. Arch. Pharm. Res. 26, 9: 727-730.
26) Thanh, P. N., Jin, W. Y., Song, G. Y., Bae, K. H., Kang S. S., 2004. Cytotoxic Coumarins from the Root of Angelica dahurica. Arch. Pharm. Res. 27, 12: 1211-1215.
27) Shin, S., Joo, S. S., Park, D., Jeon, J. H., Kim, T. K., Kim, J. S., Park, S. K., Hwang, B. Y., Kim, Y.-B., 2010. Ethanol extract of Angelica gigas inhibits croton oil-induced inflammation by suppressing the cyclooxygenase—prostaglandin pathway. J. Vet. Sci. 11, 1:43-50.
28) Widelski, J., Popova, M., Graikou, K., Glowniak, K., Chinou, I., 2009. Coumarins from Angelica lucida L.—Antibacterial Activities. Molecules. 14: 2729-2734.
29) Wedge, D. E., Klun, J. A., Tabanca, N., Demirci, B., Ozek, T., Baser, K. H. C., Liu, Z., Zhang, S., Cantrell, C. L., Zhang, J., 2009. Bioactivity-Guided Fractionation and GC/MS Fingerprinting of Angelica sinensis and Angelica archangelica Root Components for Antifungal and Mosquito Deterrent Activity. J. Agric. Food Chem. 57: 464-470.
30) Kang, S. Y., Kim, Y. C., 2007. Neuroprotective Coumarins from the Root of Angelica gigas: Structure-Activity Relationships. Arch. Pharm. Res. 30, 11: 1368-1373.
31) Deng, S., Chen, S.-N., Yao, P., Nikolic, D., van Breemen, R. B., Bolton, Judy L., Fong, H. H. S., Farnsworth, N. R., Pauli, G. F., 2006. Serotonergic Activity-Guided Phytochemical Investigation of the Roots of Angelica sinensis. J. Nat. Prod. 69, 4: 536-541.
32) Popescu, G. C., Motounu, M., Alexiu, V., 2012. Some ecophysiological and edaphic parameters of Angelica archangelica, a threatened high-altitude herb from Romanian Carpathian Mountains. Acta Hort. (ISHS) 955:303-308.
33) World Health Organization. WHO discontinues hydroxychloroquine and lopinavir/ritonavir treatment arms for COVID-19. 4 Jul. 2020. Available from: https://www.who.int/news-room/detail/04-07-2020-who-discontinues-hydroxychloroquine-and-lopinavir-ritonavir-treatment-arms-for-covid-19
34) Wilson, J. “Remdesvir gets lukewarm endorsement from experts in COVD fight”. Bloomberg, retrieved 31 Jul. 2020.

### SUMMARY OF THE INVENTION

According to a first aspect of the invention there is here provided non-toxic furocoumarin-rich extracts for use as antimicrobial agent.

The non-toxic furocoumarin-rich extract may be used as antimicrobial agent in the production of antimicrobial substances, herbal drugs or medicaments, for example, antivirals, bactericides, antifungals, antiparasitic, etc.

The provided non-toxic furocoumarin-rich extracts and related compounds and compositions may be used for treating pathogenic infections of nucleic acid-regulated microbiome including but not restricted to virus, bacteria, parasites and fungus affecting animals and human beings.

The non-toxic furocoumarin-rich extracts may be used in treating viral infections such as HIV infections, Dengue, Influenza, Hepatitis B, Hepatitis C, measles, mumps, herpes simplex, poliovirus, canine hepatitis, retrovirus, similar lentivirus, and in the treatment of current and future coronavirus infections, such as COVID-19, among others.

Furthermore, the non-toxic furocoumarin-rich extracts may be used in treating cancer of different aetiologies, such as lung cancer, pancreas cancer, leukaemia, colorectal cancer, gastric cancer, etc.

A non-toxic furocoumarin-rich extract has been proven to be effective against a culture of SARS-CoV-2 coronavirus strain in an in vitro laboratory assay, which will be described in the following sections. Therefore, furocoumarin-rich extracts are promising candidates for their use in the treatment of COVID-19. Furocoumarins are substances widely available in many plant species and therefore supplying enough effective compositions or extracts for treating all COVID-19 patients ceases to be a problem.

Preferably, the non-toxic furocoumarin-rich extracts contain at least 60% weight of furocoumarins. More preferably the non-toxic furocoumarin-rich extracts contain at least 80% weight of furocoumarins. Yet even more preferably the non-toxic furocoumarin-rich extracts contain at least 90% weight of furocoumarins.

In some embodiments, the non-toxic furocoumarin-rich extract is a purified fraction of a furocoumarin-rich plant extract and it may consist of a purified substance in excess of 90% purity.

Preferably the furocoumarin-rich extracts comprise an Angelica archangelica extract. A furocoumarin-rich Angelica archangelica extract is effective against SARS-CoV-2 coronavirus and these plants are widely distributed.

Preferably, the Angelica archangelica extract is produced from the Angelica archangelica litoralis subspecies.

Preferably the furocoumarin-rich Angelica archangelica extract is produced according to the following method:
Angelica archangelica seeds and leaves were ground to a coarse powder using a Waring blender;
495.4 g of ground plant material was subjected to Soxhlet extraction with 3500 ml of absolute ethanol during daytime for 3 days, with overnight maceration of the material in fresh ethanol each night;
10% water were added to the Soxhlet extracts and the mixture was cooled down to −20° C. and centrifuged at 10000 rpm to remove precipitated impurities;
Supernatant solution was evaporated in rotavapor to almost dryness;
Remaining oily residue was frozen and lyophilised for 3 days;
The lyophilised extract is dissolved in hexane/acetone/water (25:40:35) and after centrifugation two phases were formed, a top layer and a bottom layer;
The bottom layer is collected, filtered through a 0.45 μm membrane filter and subjected to centrifugal partitioning chromatography fractionation with a 1000 ml rotor; elution took place using (hexane/acetone/water, 25:40:35 or 31:37:32), in descending mode.

Two given ratios of solvents gave similar compositions of the two layers; the change in ratio was only done to obtain a somewhat larger volume of stationary phase. Using this system in descending mode, the most polar components elute first.

The fraction corresponding to the second peak in the resulting chromatogram was separately collected and dried under reduced pressure.

Preferably the furocoumarin-rich Angelica archangelica extract is further purified according to the following:

Further purification took place using a 250×25 mm Merck Hibar LiChrosorb (7 μm particle size) silica 60 column. The eluent for preparative HPLC was composed as follows: CHCl3/n-hexane/diethylether/ethyl acetate/water (585:400:9:6:0.4). Flow speed was 7.5 mL/min, in isocratic mode.

The furocoumarin-rich Angelica archangelica extract powder was dissolved in a suitable volume of eluent, filtered through a 0.45 μm Teflon filter and injected using a mL loop. Injection volume is 4500 μL. Fractions of max. 15 mL are collected. Fractions from overlapping peaks were collected separately and re-run. As can be seen in the example of a preparative HPLC run, the detector signal may be overloaded by the main peak. Overlapping peaks fractions were joined and separated using the same procedure in 2 runs. After every run, the column was regenerated using 20 mL of acetone, and rinsed with eluent until the baseline had returned to zero.

All fractions containing the main peak compound were mixed and dried in a rotavapor to dry powder. The yield was 450 mg.

This extraction method allowed the obtention of more than 400 mg furocoumarin-rich extract, in a purity of more than 96%, starting form Angelica archangelica seeds and leaves.

A second aspect of the invention provides a furocoumarin-rich Angelica archangelica extract produced as follows:
Angelica archangelica seeds and leaves were ground to a coarse powder using a Waring blender;
495.4 g of ground plant material was subjected to Soxhlet extraction with 3500 ml of absolute ethanol during daytime for 3 days, with overnight maceration of the material in fresh ethanol each night;
10% water were added to the Soxhlet extracts and the mixture was cooled down to −20° C. and centrifuged at 10000 rpm to remove precipitated impurities;
Supernatant solution was evaporated in rotavapor to almost dryness;
Remaining oily residue was frozen and lyophilised for 3 days;
The lyophilised extract is dissolved in hexane/acetone/water (25:40:35) and after centrifugation two phases were formed, a top layer and a bottom layer;
The bottom layer is collected, filtered through a 0.45 μm membrane filter and subjected to centrifugal partitioning chromatography fractionation with a 1000 ml rotor; elution took place using (hexane/acetone/water, 25:40:35 or 31:37:32), in descending mode.

Two given ratios of solvents gave similar compositions of the two layers; the change in ratio was only done to obtain a somewhat larger volume of stationary phase. Using this system in descending mode, the most polar components elute first.

The fraction corresponding to the second peak in the chromatogram was separately collected and dried under reduced pressure.

Preferably the furocoumarin-rich Angelica archangelica extract is further purified according to the following:

Further purification took place using a 250×25 mm Merck Hibar LiChrosorb (7 μm particle size) silica 60 column. The eluent for preparative HPLC was composed as follows: CHCl3/n-hexane/diethylether/ethyl acetate/water (585:400:9:6:0.4). Flow speed was 7.5 ml/min, in isocratic mode.

The furocoumarin-rich Angelica archangelica extract powder was dissolved in a suitable volume of eluent, filtered through a 0.45 μm Teflon filter and injected using a ml loop. Injection volume is 4500 μl. Fractions of max. 15 ml are collected. Fractions from overlapping peaks were collected separately and re-run. As can be seen in the example of a preparative HPLC run, the detector signal may be overloaded by the main peak. Overlapping peaks fractions were joined and separated using the same procedure in 2 runs. After every run, the column was regenerated using 20 ml of acetone, and rinsed with eluent until the baseline had returned to zero.

All fractions containing the main peak compound were mixed and dried in a rotavapor to dry powder. The yield was 450 mg.

This extraction method allowed the obtention of more than 400 mg furocoumarin-rich extract, in a purity of more than 96%, starting form Angelica archangelica seeds and leaves.

A third aspect of the invention provides a method of producing a furocoumarin-rich Angelica archangelica extract according to the method described in the first and second aspects of the invention.

### BRIEF DESCRIPTION OF THE DRAWINGS

Embodiments of the present invention will now be further described in the following paragraphs of this specification, by means of example only, without intending to constitute any limitation of the scope of the present invention. The following description may be better understood when read in conjunction with the attached drawings, in which:

FIG. 1 shows the cytotoxicity assay graphic representation of a stained plate.

FIG. 2 shows cellular viability of M0 macrophages, lymphocytes CD4, CD8, and B, and natural killer cells (NK and NKT) after exposure to a non-toxic furocoumarin-rich extract.

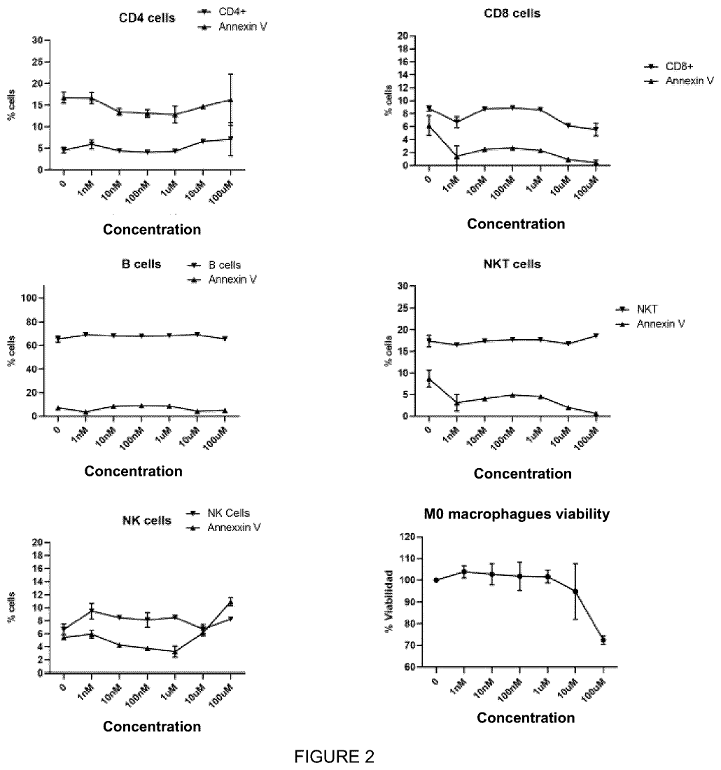

FIG. 3 shows acute and chronic C. elegans survival assay results.

### DETAILED DESCRIPTION OF THE INVENTION

Please, note that in all extracts, the average molecular weight is assumed to be that of Xanthotoxin and this is used in all concentration calculations.

### Example 1. Non-Toxic Furocoumarin-Rich Plant Extract In Vitro Assay Against SARS-CoV-2 Coronavirus

A furocoumarin-rich extract form Angelica archangelica seeds and leaves produced as previously described was tested in vitro for its antiviral efficacy against SARS-CoV-2 coronavirus using the crystal violet staining technique.

The crystal violet staining technique is based on the characteristic binding to proteins and DNA of said dye. During cell death, adherent cells in culture detach from the bottom of the culture dish, a characteristic that serves indirectly to determine cell death. Using this technique, only those cells adhered to the surface are stained. Therefore, the wells in which there are living cells will stain blue, unlike the wells in which there is a cell death process, in which the staining will be minimal or non-existent. Cellular viability testing by measuring the percentage of stained cells per well after SARS-CoV-2 exposure was carried out in triplicate, on three different periods. Three doses of the furocoumarin-rich extract were tested along with proper negative/positive controls in three different times (T1, T2 and T3).

Three (3) doses were selected based on immunotoxicology data. The raw extract was diluted in 70% ethanol. The starting point was a stock dilution of the extract in ethanol. A guide Threshold of Toxicological Concern (TTC) of similar extracts is 1.5 μg/kg of daily exposure (European Medicines Agency (EMA). Inventory of herbal substances for assessment. Herbal substances proposed to HMPC for assessment. 19 Jan. 2020. EMA/HMPC/494079/2007), so in vitro experiments were carried out taking these values into account to calculate a maximum concentration to be used.

The test experiments were performed in a final volume of 100 μl in 96-well plates. The maximum concentration to be tested was determined according to similar extracts TTC. The cells were exposed to 1.5 μg in 100 μl per well, and with an estimated molecular weight of 216.19 g/mol, the final concentration of the furocoumarin-rich extract maximum dose tested was determined to be 69 μM. According to the in-vitro study design, two additional lower concentrations (6.9 μM, 34.5 μM) of the furocoumarin-rich extract were tested as well (Example 2). The furocoumarin-rich extract doses were vehiculated, among others, with 0.15%, 0.75% and 1.5% ethanol respectively. The higher solvent concentration used was included in a cellular viability SHAM control.

Vero E6 cells (ISSA, Zaragoza, Spain) were cultured following provider descriptions. The cellular culture was maintained with a 105 cell/mL density in Vero E6 10% FBS (Sigma F7524) Dulbecco's Modified Eagle Medium (Lonza, Ref 13E12-614F) during the study. For each replication, 10 mL were cultured in 75 cm 2 Nunc EasyFlask (ThermoFisher, USA). The supernatant was discarded, and the cell monolayer was washed with 5 mL of sterile PBS. Three ml of trypsin were added, and the culture was incubated for 5 min at 37° C. with 5% CO₂ and 90% humidity. Once the cells were released, 4 mL of medium with serum were added. The cells were resuspended in a ml tube with Vero cell medium. The cells were centrifuged at 1500 rpm during 5 min, and the supernatant was discarded, and the cells were resuspended in 10 ml of complete Vero E6 medium. Fifty (50) μL of cells were mixed with 50 μL of Trypan Blue to perform cellular counting in Malassez chamber. Cellular density was adjusted to 10⁵ cells/mL by adding the necessary amount of complete Vero E6 cell medium. 100 μl/well with a 1×10⁴ Vero E6 cells density were seeded in 96-well flat-bottom plates (Nunclon Delta Surface 167008 Thermo). The plates were incubated overnight at 37° C. with 5% CO₂.

The plates were pre-incubated at 37° C. and 5% CO₂ for 1 hour. Vero E6 cells were added to columns 1, 8 and 12 as internal growing controls. 0.5%, 0.75% and 1.5% ethanol was added to columns 2, 3 and 4 as main vehicle-related toxicity control. The different furocoumarin-rich extract doses (1, 2 and 3) were added to columns 5, 6 and 7. The furocoumarin-rich extract galenic prototype (i.e. with ethanol and water as excipients) with doses 1, 2 and 3 were added to columns 9, 10 and 11 as testing wells. After 1 hour, 1 μl of cultured SARS-CoV-2 (Internal standard operation procedure—SOP-LAB 005 IT 030, WorldPathol, Spain) was added to columns 8 (positive viral control), 9, 10 and 11 (challenged wells). Moreover, 10 μl of cultured SARS-CoV-2 (Internal SOP LAB 005 IT 030, WorldPathol, Spain) was added to column 12 (10-fold positive viral control). The plates were incubated for 72 hours at 37° C. and 5% CO₂ after viral exposure.

The viral agent was a high-pathogenic strain of severe acute respiratory syndrome coronavirus 2 (SARS-CoV-2) isolated and cultured from a 72-years old patient at University Clinical Hospital Lozano Blesa (Zaragoza, Spain). The second-passage vials with the SARS-CoV-2 strain were provided by Dr Julian Pardos (ISSA, UNATI, Zaragoza, Spain). The coronavirus was maintained and cultured following UNATI protocols in level 3 biosafety (BSL3) facilities at Zaragoza (Spain) (WorldPathol, Zaragoza, Spain). The Tissue Culture Infectious Dose 50% (TCID50) was determined to be 1.47×10⁶/ml.

The crystal violet staining of attached, living cells was performed after incubation with the virus. The plates were washed two times with PBS. 200 μl of formaldehyde 4% (Panreac 252931.1212) were added to each well and incubated for 1 hour at room temperature. Subsequently, 50 μl of staining solution (0.5% crystal violet and 20% methanol) were added per well (Sigma, C0775) (Panreac 131091.1212). After incubation for 15 min at room temperature, the plates were washed four times with water.

The cellular viability (living cells) was directly observed by inverted microscope (DM IL LED Leica). A strong positive cellular blue staining was considered as viable cells (when more than 75% of well is stained). Intermediate or weak cellular staining was considering as unviable cells (dead cells) (less than 75% of well is stained). Counting was performed by two different technologists per experiment.

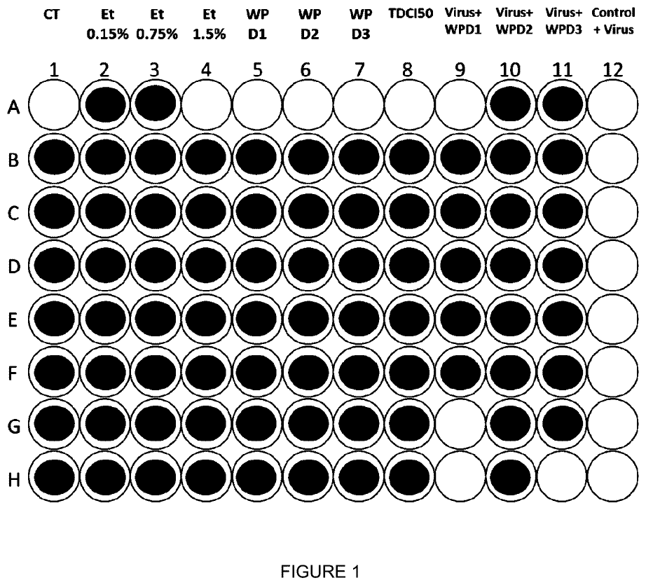

FIG. 1 shows data collected on 27/07/2020 (first replicate). The columns are divided as previously indicated in experimental design. The black coloured dots represent viable cells (living cells) and the white coloured dots represent unviable cells (dead cells). It is to be noted that this replica had a TCDI50 control well (not kill 50% of Vero E6 cells). This example is shown to highlight the marked cytoprotective antiviral effect of the furocoumarin-rich extract in D2 and D3 columns comparing to 10-folds viral load positive control (100% cellular death) against in vitro cultured cells exposure to SARS-CoV-2.

All data were analysed by Microsoft® Excel® STATS (Microsoft 365 MSO—16.0.13231.20110-32 bits, ID 00265-80196-36405-AA936). The results were presented as Mean±SD (Standard Deviation). One-way ANOVA was used to confirm statistical difference of multiple groups between treated and no treated groups. The furocoumarin-rich extract—TCD50 groups were analysed by two-sample t-test assuming equal variances to confirm significant differences. *P<0.05, **P<0.01 and ***P<0.001. P<0.05 was considered as significant.

Results

The descriptive statistics are shown in Table 1. The maximum standard deviation was observed in the TCDI50 group, mostly due to outlier results in the firsts replications of the experiment (FIG. 1). After that, more coherent and consistent TCID50 results (5±1.73) were obtained in the rest of replications.

A marked significant difference between groups was found for at least one group as stated by analysis of variance of a factor ANOVA (furocoumarin-rich extract treatment) (Table 2). No differences were found by analysing the effect of solvents or raw material vs the Vero culture in a two-sample t-test assuming equal variances (Table 3).

A marked increase of cell viability when comparing to TCDI50 control was found in SARS-CoV-2-infected groups treated with furocoumarin-rich extract, corresponding to 34.5 μM (WP2) and 69 μM (WP3) doses (Table 1). Significant differences were found between such groups by analysing two-sample t-test assuming equal variances as well, confirming preliminary descriptive results. Following these results, ANOVA significancy can be checked directly to evidence a significant furocoumarin-rich extract treatment cytoprotective effect.

### CONCLUSIONS

The Angelica archangelica furocoumarin-rich extract has shown a marked significant in vitro cytoprotective effect in SARS-CoV-2-exposed Vero E6 cellular cultures by using 34.5 and 69 μM doses, corresponding to maximum TTC of similar compounds. Remarkably, a high-virulent SARS-CoV-2 strain has been used during the experiments so, we can postulate that furocoumarin-rich extracts are a promising potential treatment for COVID19.

These results may rule out in vitro toxicity of solvents and raw material in the proposed model. It is worth to mention that the furanocoumarin-rich extracts may be phototoxic and may affect cellular viability in studied doses.

TABLE 1
Descriptive statistics of in vitro SARS-CoV-2 exposure test. The furocoumarin-rich extract doses
are 6.9 (WP1), 34.5 (WP2) and 69 (WP3) μM per well. Different ethanol concentrations (15%,
50%, 75%) were added as SHAM control. TCDI50 is considered positive control and the main infective
group to be compared with treatment. TCDI50-SARS 10-fold is considered maximum infective positive
internal control (cell death). CT group was composed by non-treated Vero E6 2% FSB medium culture
and was considered treatment-negative control (maximum cell viability).

| | | | | | | | | | SARS + | SARS + | SARS + | SARS |
|---|---|---|---|---|---|---|---|---|---|---|---|---|
| | CT | Et_0.15 | Et_0.5 | Et_0.75 | WP1 | WP2 | WP2 | TCDI50 | WP1 | WP2 | WP3 | 10-f |
| Mean | 8 | 8 | 7.33 | 7.67 | 7.33 | 7.33 | 7.67 | 5 | 7.00 | 7.67 | 7.67 | 0 |
| Standard | 0 | 0 | 0.33 | 0.33 | 0.33 | 0.33 | 0.33 | 1 | 0.58 | 0.33 | 0.33 | 0 |
| error | | | | | | | | | | | | |
| Median | 8 | 8 | 7 | 8 | 7 | 7 | 8 | 4 | 7 | 8 | 8 | 0 |
| Mode | 8 | 8 | 7 | 8 | 7 | 7 | 8 | 4 | 7 | 8 | 8 | 0 |
| Standard | 0 | 0 | 0.58 | 0.58 | 0.58 | 0.58 | 0.58 | 1.73 | 1 | 0.58 | 0.58 | 0 |
| deviation | | | | | | | | | | | | |
| Variance | 0 | 0 | 0.33 | 0.33 | 0.33 | 0.33 | 0.33 | 3 | 1 | 0.33 | 0.33 | 0 |

TABLE 2
Analysis of variance of a factor (extract treatment) of in vitro
SARS-CoV-2 exposure test. Null hypothesis was considered as
no observed differences between groups. *p < 0.05, **p < 0.01
and ***p < 0.001. p < 0.05 was considered to be significant.

| Origin of variations | F | Probability | Critical value for F |
|---|---|---|---|
| Between groups | 29.03349282 | 0.000*** | 2.216308646 |

TABLE 3
Two-sample t-test assuming equal variances results of in vitro SARS-CoV-2
exposure test for solvents and raw material. Non-treated Vero E6 2% medium
culture (CT) group was considered treatment-negative control (maximum
cell viability) for comparison (data no shown). Ethanol 0.15%, 0.75%
and 1.5% respectively were considered as solvent control. The furocoumarin
rich extract doses were 6.9 μM (WP1), 34.5 μM (WP2) and 69 μM (WP3)
and considered as extract-treated non-SARS-CoV-2-exposed groups.

| | Et_0.15 | Et_0.5 | Et_0.75 | WP1 | WP2 | WP3 |
|---|---|---|---|---|---|---|
| Mean | 8.0000 | 7.3333 | 7.6667 | 7.3333 | 7.3333 | 7.6667 |
| Variance | 0.0000 | 0.3333 | 0.3333 | 0.3333 | 0.3333 | 0.3333 |
| t Stat | 65535 | −2 | −1 | −2 | −2 | −1 |
| P(T <= t) one-tail | # [symbol] NUM! | 0.0581 | 0.1870 | 0.0581 | 0.0581 | 0.1870 |
| t Critical one-tail | 2.1318 | 2.1318 | 2.1318 | 2.1318 | 2.1318 | 2.1318 |
| P(T <= t) two-tails | # [symbol] NUM! | 0.1161 | 0.3739 | 0.1161 | 0.1161 | 0.3739 |
| t Critical two-tails | 2.7764 | 2.7764 | 2.7764 | 2.7764 | 2.7764 | 2.7764 |

# [symbol] NUM! Both groups behave equal so, no comparison could be raised up.
* p < 0.05, ** p < 0.01 and *** p < 0.001. p < 0.05 was considered to be significant.

TABLE 4
Two-sample t-test assuming equal variances results of in vitro
SARS-CoV-2 exposure test for SARS-CoV-2-infected extract-treated
groups. TCDI50 SARS-CoV-2-infected cell death-positive group was
considered for comparison (data no shown). *p < 0.05, **p < 0.01
and ***p < 0.001. p < 0.05 was considered to be significant.
Furocoumarin rich extract doses were 6.9 μM (WP1), 34.5 μM
(WP2) and 69 μM (WP3). Tissue Culture Infectious Dose 50%
(TCID50) of SARS-CoV-2 (SARS) was determined to be 1.47 × 106/ml.

| | SARS + WP1 | SARS + WP2 | SARS + WP3 |
|---|---|---|---|
| Mean | 7 | 7.667 | 7.667 |
| Variance | 1 | 0.333 | 0.333 |
| t Stat | 1.732 | 2.530 | 2.530 |
| P(T <= t) one-tail | 0.079 | 0.032* | 0.032* |
| t Critical one-tail | 2.132 | 2.132 | 2.132 |
| P(T <= t) two-tails | 0.158 | 0.065 | 0.065 |
| t Critical two-tails | 2.776 | 2.776 | 2.776 |

### Example 2. Cytotoxicity Assays of a Furocoumarin-Rich Plant Extract

A furocoumarin-rich plant extract toxicity was assessed at different concentrations in immune system cells. B6-mouse-derived bone marrow monocytes (BMC) were differentiated to M0 macrophages and incubated for 24 h with different extract doses. Proper extract-negative and highest concentration-used diluent (SHAM) controls were included for comparison. After 24 hours, cell viability was determined by using the PrestoBlue assay. In addition, macrophage inflammatory response was determined by measuring IL6 and TNFα expression in supernatant. On the other hand, B6-mouse-derived splenic cells (SC) were manually disintegrated and washed. Afterwards, erythrocytes were lysed and SC were counted for seeding 1×10⁶ cells/mL of medium. Treated SC were incubated with the same concentrations of the extract used for macrophages. After 24 hours they were collected, washed and labelled with: Annexin V-PE to quantify dead cells; CD3-FITC, CD8-APC, CD4-vioBlue and NK1.1-APCvio770 to detect and quantify NK, NKT, CD4 T and CD8 T cells; Annexin V-APC, CD19-PE and CD3-FITC to detect and quantify lymphocytes B. Final results were obtained by flow cytometry.

The extract was produced as follows:

700 g of seeds and roots of Angelica archangelica var. litoralis were coarsely comminated. The plant-derived material was successively extracted for 36 h with 15 L of methanol in a Soxhlet extractor. The extracts were each concentrated under reduced pressure in a rotary evaporator. The extracts were purified with high-performance liquid chromatography to reach 98% purity (UPLC High-performance Liquid Chromatographer XEVO TQD, Waters, US). A standard dose was determined considering the previous toxicologic data from the raw extract plant (European Medicines Agency (EMA). Inventory of herbal substances for assessment. Herbal substances proposed to HMPC for assessment. 19 Jan. 2020. tEMA/HMPC/494079/2007). Ethanol was selected as the most appropriate solvent, according to previous chemical characterization studies. EMEA propose a risk management TTC (Threshold of Toxicology Concern) is 1.5 mg/day.

The raw material was diluted in 70% ethanol at concentrations of 1 nM, 10 nM, 100 nM, 1 μM, 10 μM and 100 μM. Higher concentration-used solvent was used as SHAM control of cellular viability. Macrophages and immune cells were incubated and tested in these doses.

For in vivo studies 1 mM dose was added to acute and chronic studies (10.000 times higher than reference dose). For these studies, a 25° C.-cultivated infertile strain of C. elegans was used. Worms were seeded and incubated with the extract for proper times.

Obtaining Stem Cells from Bone Marrow

A minimal-disease certified mouse (B6, Charles River, US) was killed by dislocation. The abdomen and hind legs were sterilized with 70% ethanol, and femurs and tibias were dissected from body. Bones were carefully cleaned with ethanol and placed in a ml skirt containing medium to remove ethanol. Complete dissection and removal of muscular tissue was performed in a Petri plate. Bones were transferred to a plate containing ethanol for sterilization by contact. After 1 min, bones were transferred to a plate containing medium to remove the ethanol.

Under sterile conditions, cleaned bones were transferred to another plate with 5 mL of medium in which the bone marrow cells (BMC) were eluted by injecting 2 mL of DMEM or RPMI medium through the bone marrow cavity. Medium was resuspended until a homogeneous suspension was obtained and filtered. Erythrocytes were lysed, then, supernatant was removed by centrifugation at 1200 rpm for 5 min. Recovered bone marrow cells were resuspended in 10 mL of BMDM medium. An aliquot was taken for a 1:10 dilution (100 μl of cell suspension and 900 μl of medium) and counted with trypan blue in a Neubauer chamber. Final BMDM suspension was adjusted to a cell concentration of 1×10⁶ cells/ml.

M0 Macrophage Differentiation and Cell Viability

Ten ml of the BMDM-based BMC suspension were seeded in 100 mm Petri dishes and incubated at 37° C. and 5% CO₂. On the third day, the supernatant was removed and ml of fresh BMDM medium was added. This operation was repeated on the sixth day. On seventh day, BMC are harvested for planting in 96-well plates. BMC were resuspended in 5 mL of DMEM 10% FBS and adjusted the concentration to 5×10⁵ cells/ml. One hundred (100) μl of the previous suspension was seeded in 96-well plates. Nine hundred μl of DMEM 10% FBS were added each well, adjusting for a final concentration of 5×10⁴ cells per well. Plates were incubated for 24 hours at 37° C. and 5% CO₂.

After 24 hours, WP2006001 doses were added by making a serial dilution 1/10 and incubated for another 24 h.

Obtaining Lymphocytes from the Spleen

A minimal-disease certified mouse (B6, Charles River, US) was killed by dislocation. Its abdomen was sterilized with rising 70% ethanol. Spleen was carefully extracted and crushed through a cell strainer. Splenic cells (SC) were washed with RPMI and centrifuged at 1200 rpm for 5 min. SC were counted and adjusted to 1×10⁶ cells/ml.

Cellular Viability by PrestoBlue Assay

Cell viability was analysed by Prestoblue HS assay. PrestoBlue HS (high sensitivity) contains resazurin and a proprietary buffering system (#P50200, ThermoFisher, US). When added to media, the PrestoBlue reagents are rapidly taken up by cells. The reducing environment within viable cells converts the non-toxic resazurin in the PrestoBlue reagent to an intensely red-fluorescent dye. This change can be detected by measuring fluorescence or absorbance.

The cells were added in appropriate medium to microplate wells. Nine hundred μl of BCM or SC and 100 μl of PrestoBlue HS reagent were added in 96-well plates. The plates were incubated at 37° C. for 10 minutes. The absorbance was measured by using iMark™ Microplate Absorbance Reader (BioRad, Germany). The signal was stable for 7 hours after incubation.

Lymphocytes Differentiation by Flow Cytometry

The treated SC were incubated with the same concentrations of the extract used for macrophages. After 24 hours they were collected, washed and labelled with: Annexin V-PE to quantify dead cells; CD3-FITC, CD8-APC, CD4-vioBlue and NK1.1-APCvio770 to detect and quantify natural killer (NK), natural killer cytotoxic (NKT), CD4 T and CD8 T cells; Annexin V-APC, CD19-PE and CD3-FITC to detect and quantify lymphocytes B. All cells were staining following manufacturer's instructions without changes. Number of stained cells were analysed (405, 488 y 635 nm) by using flow cytometry (Flow cytometer GALLIOS, Beckman Coulter) following manufacturer's protocol without changes.

In Vivo Toxicity: Caenorhabditis elegans Assay

C. elegans Strain

The C. elegans strain used was the glp-4 mutant. Caenorhabditis elegans gene glp-4 was identified by the temperature-sensitive allele bn2 where mutants raised at the restrictive temperature (25° C.) produce adults that are essentially germ cell deficient C. elegans.

Culturing and Synchronization

C. elegans worms were propagated on NGM agar plates with kanamycin 50 μg/ml and streptomycin 100 μg/ml at 20° C. (NGM Lite, US Biological Life Sciences, Swampscott in Massachusetts, USA) using E. coli OP50 as source of food. Due to the presence of worms at different developmental stages in cultures, these must be synchronized for further use. Synchronization process consisted of on killing larvae and adult worms and the debilitation of C. elegans cuticle through a bleaching solution to release eggs from gravid worms. When eggs were obtained from synchronization, a pool of similar aged and developed worms were obtained.

Three 100 mm NGM agar plates were washed with 6 mL of M9 buffer (Na2HPO4, 6 g; NaCl, 5 g; KH₂PO₄, 3 g; distilled H₂O, 1 L; and 1 ml of MgSO₄ 1 M) each one. Worm suspension in M9 buffer was transferred to 15 ml Falcon tubes. Four ml more of M9 buffer can be used to unstick eggs from NGM agar plates. Worm suspension was centrifuged in a commercial centrifuge at 650×g for 2 minutes. The supernatant was discarded until 2 ml remained in the 15 ml Falcon tube. Bleaching solution containing NaOH and NaClO was added to the worms to reach a final volume of 4 ml and final NaOH and NaClO concentrations of 0.25 M and 1% respectively. NaClO solution must be prepared daily due to loss of power. Commercial bleach apt for water treatment can be used. Worms with bleaching solution were vortexed for ten seconds and an aliquot was taken to check the worms under a dissecting microscope. If worms kept alive or cuticle of most worms had not been broken, more 10 seconds vortex evented up to a maximum contact time of 6 minutes was required. Higher concentration of NaOH could be needed to get the breakage of the cuticle if suspension contained too many worms. Watching worms regularly on a dissecting microscope provided the best information to know when the bleaching process should be stopped. Once previous conditions were reached, the 15 mL Falcon tube was filled with M9 buffer and centrifuged 2 minutes at 650×g and repeated 2 times more to reduce the amount of NaOH and NaClO remaining. Supernatant of washing M9 was discarded until only 0.5 ml stayed in the tube.

Eggs pellet was resuspended and plated on a NGM agar plate without E. coli OP50 (ISSA, Zaragoza, Spain) to allow eggs to hatch and reduce developmental differences in new larvae due to different age of eggs. E. coli OP50 was added to the NGM agar plate 24 h later.

Acute Survival Assay

L1 larvae obtained from synchronization were cultured at 25° C. until worms developed to L4 stage. L4 worms were harvested from plates and washed 3 times with M9.

Approximately 15 worms per well were placed in a 96-well flat bottom microtiter plate. A total of 45 worms (3 wells) were assessed by each dose of the extract. Worms without treatment served as negative controls. Survival assays were carried out for a day at 25° C. in three different periods.

Chronic Survival Assay

L1 larvae obtained from synchronization were cultured at 25° C. until worms developed to L4 stage. L4 worms were harvested from plates and washed 3 times with M9. Approximately 15 worms per well were placed in a 96-well flat bottom microtiter plate. A total of 45 worms (3 wells) were assessed by the extract doses. Worms without treatment served as negative controls. Survival assays were carried out for 21 days at 25° C. in three different periods. Every 7 days the extract and bacteria that serve as food were added to the C. elegans. Chronic toxicity worms were seeded and counted twice a week calculating the percent of worms that survived at each moment with respect to the beginning of the experiment.

Graphs and Interpretation

Lymphocytes CD4, CD8, B and natural killer cells (NK and NKT) coming from SC and M0 macrophages coming from BMC were analysed in vitro for toxicology assessment. Cytotoxicity data were plotted in curves of relative fluorescence units (markers) vs. the extract doses to generate quantitative results. Interpretations were made by direct comparison between expected cellular viability and observations.

Worms survival rates between the extract doses were plotted for 24 hours (acute challenge) and 3, 7, 10, 14, 17 and 21 days (chronic challenge). Worms survival was monitored with a dissecting microscope. Results were presented as mean±SD (Standard Deviation) of survival worms during the experiment. Interpretations were made by direct comparison between expected worm survival and observations.

Results

Cytotoxicity of Lymphocytes B, T, Natural Killer Cells (NK and NKT) and Macrophages

No toxicity of the tested extract doses on CD4 and CD8 T cells or on B cells and NKT was observed (Tables 5, 6, 7).

TABLE 5
Annexin V staining of lymphocytes B after furocoumarin-rich
extract exposure. Number of stained cells were analysed (405,
488 y 635 nm) by using flow cytometry (Flow cytometer
GALLIOS, Beckman Coulter) following manufacturer's
protocol without changes.

| | Annexin V | |
|---|---|---|
| Name of the data file | on B Cells | B cells |
| B CELLS 0 | 7.33 | 63.44 |
| B CELLS 1 nM | 3.64 | 68.66 |
| B CELLS 10 nM | 8.64 | 68.23 |
| B CELLS 100 nM | 8.18 | 66.79 |
| B CELLS 1 uM | 8.65 | 68.26 |
| B CELLS 10 uM | 3.49 | 69.33 |
| B CELLS 100 uM | 5.07 | 65.71 |
| B CELLS 0 | 6.84 | 67.28 |
| B CELLS 1 nM | 3.54 | 69.54 |
| B CELLS 10 nM | 8.37 | 68.46 |
| B CELLS 100 nM | 9.96 | 69.17 |
| B CELLS 1 uM | 8.62 | 68.57 |
| B CELLS 10 uM | 5.01 | 69.06 |
| B CELLS 100 uM | 4.65 | 65.3 |

However, increased annexin V staining meaning slight decrease of cellular viability was observed on NK cells starting in 10 μM towards to 100 μM doses. Such effect was determined on macrophages by reducing cell viability by 30% after exposure to 100 μM of the extract as well (Table 5).

Remarkably, the extract did not induce an inflammatory response in M0 macrophages, which we were able to verify by released IL6 and TNFα after challenge of all doses (Table 6). IL6 and TNFα values are very low and in some samples are very close to or even below the limit of detection (detection limit IL6: 4 μg/ml, TNFα: 8 μg/ml).

TABLE 6
Annexin V, CD4+, CD8+, NK+ and NKT+ staining of lymphocytes T and NK after furocoumarin-
rich extract exposure. Number of stained cells were analysed (405, 488 y 635 nm) by using flow
cytometry (Flow cytometer GALLIOS, Beckman Coulter) following manufacturer's protocol without changes.

| | Annexin V on | Annexin V on | Annexin V on | Annexin V on | | | | |
|---|---|---|---|---|---|---|---|---|
| Data set | CD4+ Cells | CD8+ Cells | NK Cells | NKT Cells | CD4+ | CD8+ | NK | NKT |
| T NK CELLS 000 | 17.66 | 7.25 | 5.6 | 10.12 | 4.09 | 9.09 | 7.28 | 18.37 |
| 00063130.715 | | | | | | | | |
| T NK CELLS 001 | 17.56 | 2.55 | 6.4 | 4.52 | 5.21 | 7.33 | 10.34 | 16.55 |
| 00063137.722 | | | | | | | | |
| T NK CELLS 010 | 14.02 | 2.66 | 4.22 | 4.13 | 4.4 | 8.83 | 8.72 | 17.77 |
| 00063136.721 | | | | | | | | |
| T NK CELLS 100 | 13.76 | 2.96 | 3.74 | 4.71 | 4.3 | 9.1 | 8.95 | 17.83 |
| 00063135.720 | | | | | | | | |
| T NK CELLS 001 | 14.21 | 2.22 | 3.86 | 4.41 | 4.44 | 8.87 | 8.62 | 17.72 |
| 00063134.719 | | | | | | | | |
| T NK CELLS 010 | 14.29 | 1.21 | 6.45 | 2.16 | 6.9 | 6.01 | 7.24 | 16.89 |
| 00063133.718 | | | | | | | | |
| T NK CELLS 100 | 20.47 | 0.76 | 11.42 | 0.63 | 4.41 | 6.25 | 8.33 | 18.68 |
| 00063132.717 | | | | | | | | |
| T NK CELLS 000 | 15.79 | 5.07 | 5.29 | 7.33 | 5.07 | 8.51 | 6.1 | 16.41 |
| 00063139.724 | | | | | | | | |
| T NK CELLS 001 | 15.72 | 0.26 | 5.52 | 1.77 | 6.68 | 6.11 | 8.63 | 16.46 |
| 00063146.731 | | | | | | | | |
| T NK CELLS 010 | 12.92 | 2.37 | 4.34 | 3.98 | 4.45 | 8.64 | 8.26 | 17 |
| 00063145.730 | | | | | | | | |
| T NK CELLS 100 | 12.47 | 2.46 | 3.79 | 5.24 | 3.87 | 8.69 | 7.35 | 17.54 |
| 00063144.729 | | | | | | | | |
| T NK CELLS 001 | 11.45 | 2.44 | 2.65 | 4.72 | 4.24 | 8.38 | 8.44 | 17.63 |
| 00063143.728 | | | | | | | | |
| T NK CELLS 010 | 15.04 | 0.67 | 5.82 | 1.85 | 6.31 | 6.27 | 6.23 | 16.65 |
| 00063142.727 | | | | | | | | |
| T NK CELLS 100 | 12.08 | 0.19 | 10.51 | 0.65 | 9.85 | 4.86 | 8.18 | 18.51 |
| 00063141.726 | | | | | | | | |

In Vivo Toxicity of the Extract

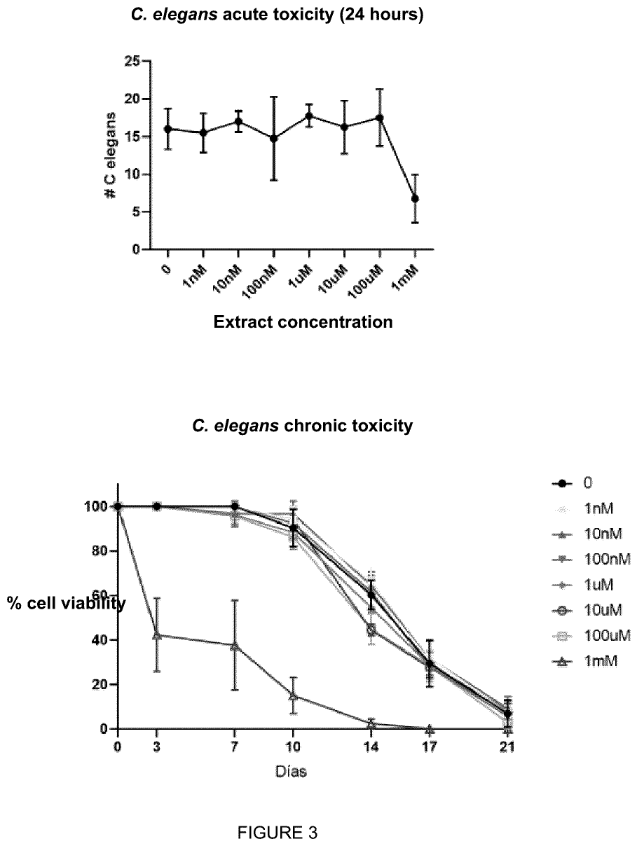

In vivo studies were performed in C. elegans (FIG. 3). Using this model, a 24-hour acute toxicity study and a chronic toxicity study at 21 days were carried out. No toxicity was observed on 1 nM, 10 nM, 100 nM, 1 μM, 10 μM and 100 μM in both assays. SHAM controls of these doses did not show any toxicity related to solvent in both assays.

In both assays, only the highest dose showed toxicity (1 mM), which is presumably due to the higher ethanol concentration (20%), such as stated by its own SHAM control.

TABLE 7
IL6 and TNFα analyses from M0 macrophages supernatant.

| | | | IL6 | | average | TNFα | | average | |
|---|---|---|---|---|---|---|---|---|---|
| M1 | control− | | 4.8 | 5.52 | 5.16 pg/ml | 20.52 | 28.96 | 24.74 | pg/ml |
| M2 | 0 | | 4.48 | 5.28 | 4.88 pg/ml | 17.08 | 28.32 | 22.7 | pg/ml |
| M3 | 1 | nM | 4.6 | 5.28 | 4.94 pg/ml | 19.16 | 25.84 | 22.5 | pg/ml |
| M4 | 10 | nM | 4.4 | 5.8 | 5.1 pg/ml | 17.48 | 32.52 | 25 | pg/ml |
| M5 | 100 | nM | 4.48 | 6.96 | 5.72 pg/ml | 21 | 38.2 | 29.6 | pg/ml |
| M6 | 1 | uM | 5.04 | 0.12 | 2.58 pg/ml | 24.68 | 0.04 | 12.36 | pg/ml |
| M7 | 10 | uM | 7.12 | 0.16 | 3.64 pg/ml | 29.64 | 0.76 | 15.2 | pg/ml |
| M8 | 100 | uM | 5.16 | 0.24 | 2.7 pg/ml | 26.44 | 0 | 13.22 | pg/ml |

| | | | | | | | | |
|---|---|---|---|---|---|---|---|---|
| M9 | solvent | 5.52 | 0.12 | 2.82 pg/ml | 27.04 | 0.36 | 13.7 | pg/ml |

| | | | |
|---|---|---|---|
| Control + | LPS | 605.44 pg/ml | 1236.12 pg/ml |

### CONCLUSIONS

Taken together cytotoxicity and in vivo assays data we postulate that the furocoumarin-rich extract described has shown very few or none evidence of toxicity. NK and macrophages showed slight decrease of viability on the highest dose, probably due to the same effect of solvent stated on in vivo assays.

It is worthy to mention that the reference dose for proof-of-concept manufacturing of the extract as potential treatment for COVID19 is 100-folds lower than toxicity threshold observed in these studies.

1. A non-toxic furocoumarin-rich extract for use as antimicrobial agent.

2. A non-toxic furocoumarin-rich extract according to claim 1 wherein it is used as antimicrobial agent in the production of antimicrobial substances, herbal drugs or medicaments.

3. A non-toxic furocoumarin-rich extract according to claim 2 wherein it used for treating pathogenic infections of nucleic acid-regulated microbiome including but not restricted to virus, bacteria, parasites and fungus affecting animals, mammals and human beings.

4. A non-toxic furocoumarin-rich extract according to claim 2 wherein it used for the treatment of viral infections such as HIV infections and infections of flavivirus (among others, Dengue), Influenza, Hepatitis B, Hepatitis C, measles, mumps, herpes simplex, poliovirus, canine hepatitis, paramyxovirus, retrovirus, similar lentivirus, phlebovirus (among others Rift Valley Fever virus), and in the treatment of current coronavirus infections, such as COVID-19, further Klebsiella pneumoniae, Yersinia enterocolitica, Escherichia coli, Pseudomonas aeruginosa, Salmonella choleraesuis, Enterobacter cloacae, Serratia marcescens, Staphylococcus epidermidis, Staphylococcus aureus, Streptococcus pyogenes, Listeria monocytogenes, Corynebacterium minutissimum, Bacillus cereus (vegetative), Lactobacillus species, Bifidobacterium adolescentis, Propionibacterium acnes, Clostridium perfringens (vegetative), Treponema pallidum, Borrelia burgdorferi, HIV-1 (cell associated), HTLV-I, HTLV-II, CMV, WNV, DHBV (model for HBV), BVDV (model for HCV), Pseudorabies (model for CMV), Chikungunya, Dengue, Influenza A, Bluetongue virus, Adenovirus, Protozoa, Trypanosoma cruzi, Plasmodium falciparum, Babesia microti, T-Cells/Leukocytes, T-Cells.

5. A non-toxic furocoumarin-rich extract according to claim 2 wherein it used for the treatment of cancer of different aetiologies.

6. A non-toxic furocoumarin-rich extract according to claim 2 wherein it used for inflammatory pathways modulation.

7. A non-toxic furocoumarin-rich extract according to any previous claim comprising at least 60% weight of furocoumarins, preferably at least 80% weight of furocoumarins, and more preferably at least 90% weight of furocoumarins.

8. A non-toxic furocoumarin-rich extract according to any previous claim wherein the non-toxic furocoumarin-rich extract is a purified fraction of a furocoumarin-rich plant extract.

9. A non-toxic furocoumarin-rich extract according to any previous claim consisting of a purified complex of furocoumarins in excess of 90% purity, comprising a main proportion of a xanthotoxin-like molecule with the molecular weight of 217.3±2 Da and a minor proportion comprising one or more of the following groups of furocoumarins: imperatorin, phellopterin, bergapten, isopimpinellin and oxypeucedanin and phellopterin.

10. A non-toxic furocoumarin-rich extract according to any previous claim consisting of a purified complex of furocoumarins in excess of 90% purity, comprising a main proportion of xanthotoxin and a minor proportion comprising one or more of the following furocoumarins: imperatorin, phellopterin, bergapten, isopimpinellin and oxypeucedanin.

11. A non-toxic furocoumarin-rich extract according to any previous claim comprising an Angelica archangelica extract, preferably from the arctic Angelica variety.

12. A non-toxic furocoumarin-rich extract according to claim 11 comprising an extract from the Angelica Subspecies Archangelica and Subspecies Litoralis.

13. A non-toxic furocoumarin-rich extract according to claim 11 or claim 12 produced according to the following method steps:
Angelica archangelica seeds and/or leaves were ground to a powder;
The ground plant material was subjected to Soxhlet extraction with absolute ethanol during daytime for 3 days, with overnight maceration of the material in fresh ethanol each night;
Addition of 10% water were to the Soxhlet extracts and cooling of the mixture was cooled down to −20° C. and centrifuged at 10.000 rpm to remove precipitated impurities;
Evaporation of supernatant solution under reduced pressure to almost dryness;
Freezing and lyophilisation of remaining oily residue for 3 days;
Dissolution of the lyophilised extract is in hexane/acetone/water (25:40:35) and then centrifugation where two phases form, a top layer and a bottom layer;
Collection of the bottom layer, filtration through a 0.45 μm membrane filter and centrifugal partitioning chromatography fractionation with a 1000 mL rotor; elution using (hexane/acetone/water, 25:40:35 or 31:37:32), in descending mode;
Separate collection of the fraction corresponding to the second peak in the resulting chromatogram and drying under reduced pressure.

14. A non-toxic furocoumarin-rich extract according to claim 13 further purified according to the following:
Further purification using a 250×25 mm Merck Hibar LiChrosorb® (7 μm particle size) silica 60 column with a CHCl3/n-hexane/diethylether/ethyl acetate/water (585:400:9:6:0.4) eluent and a flow speed of 7.5 mL/min in isocratic mode; and
Dissolution of the resulting furocoumarin-rich Angelica archangelica extract powder in a suitable volume of eluent, filtration through a Teflon® filter, injection in a preparative chromatograph and collection of all fractions containing the main peak, mixing thereof and drying in a rotavapor to dry powder.

15. A non-toxic furocoumarin-rich Angelica archangelica extract produced according to claims 13 and 14, preferably from the arctic Angelica variety.

16. A method of producing a furocoumarin-rich Angelica archangelica extract according to claims 13 and 14.

17. A non-toxic nucleic acid cross linking agent, wherein said nucleic acid cross-linking agent is isolated from a non-toxic furocoumarin-rich extract according to claims 1 to 16.

18. A non-toxic nucleic acid cross linking agent according to claim 17 wherein the non-toxic nucleic acid cross-linking agent is a xanthotoxin-like molecule.

19. A non-toxic nucleic acid cross linking agent according to claim 18 wherein the xanthotoxin-like molecule has a molecular weight of 217.3+/−2DA.

20. A non-toxic nucleic acid cross linking agent according to claim 19 wherein the xanthotoxin-like molecule is omitting the pyran and/or the furan ring and/or the methoxy group in position 9 is substituted by one of the following: Mg (magnesium), Cl (chlorine), Na (sodium), K (calcium), Ca (calcium), F (fluoride), SiO (silicon monoxide), CO₂ (carbon dioxide), SO4 (sulfate), H2S (hydrogen sulphite) and can be also O (oxygen), S (sulphur), P (phosphorus), Br (bromine), I (iodine), Li (lithium).

21. A pharmaceutical composition comprising a nucleic acid cross-linking agent according to any of claims 17 to 20 and a physiologically tolerable carrier or excipient, a wherein said nucleic acid cross-linking agent is selected from the group of non-toxic furanocoumarins consisting of: xanthotoxin-like molecule, imperatorin, phellopterin, bergapten, isopimpinellin, oxypeucedanin, isoimperatorin and archangelicin.

22. The composition of claim 21 wherein said nucleic acid cross-linking agent xanthotoxin-like molecule is omitting the pyran and/or the furan ring and/or the methoxy group in position 9 is substituted by one of the following: Mg (magnesium), Cl (chlorine), Na (sodium), K (calcium), Ca (calcium), F (fluoride), SiO (silicon monoxide), CO₂ (carbon dioxide), SO4 (sulfate), H2S (hydrogen sulphite) and can be also O (oxygen), S (sulphur), P (phosphorus), Br (bromine), I (iodine), Li (lithium).

23. The composition of claim 21 or 22 wherein the nucleic acid cross-linking agent is a non-toxic molecule with formula C17H16O6.

24. A conjugate of hemoglobin and a non-toxic nucleic acid cross linking agent, wherein said nucleic acid cross-linking agent is isolated from the furocoumarin-rich non-toxic extract according to claims 1 to 16.

25. The hemoglobin conjugate of claim 24, wherein said non-toxic nucleic acid cross-linking agent is a xanthotoxin-like molecule.

26. The haemoglobin conjugate of claim 25 wherein said xanthotoxin-like molecule has a molecular weight of 217.3+/−2DA.

27. The haemoglobin conjugate of claim 26, wherein said xanthotoxin-like molecule is part of a composition.

28. A synthetic pharmaceutical composition comprising a conjugate of haemoglobin and a nucleic acid cross-linking agent together with a physiologically tolerable carrier or excipient, a wherein said nucleic acid cross-linking compound is selected from the group of non-toxic furanocoumarins consisting of: xanthotoxin-like molecules, imperatorin, phellopterin, bergapten, isopimpinellin and oxypeucedanin.

29. The composition of claim 28, wherein said nucleic acid compound cross-linking agent is omitting the pyran and/or the furan ring and/or the methoxy group in position 9 is substituted by one of the following: Mg (magnesium), Cl (chlorine), Na (sodium), K (calcium), Ca (calcium), F (fluoride), SiO (silicon monoxide), CO2 (carbon dioxide), SO4 (sulphate), H2S (hydrogen sulphite) and can be also (oxygen), S (sulphur), P (phosphorus), Br (bromine), I (iodine), Li (lithium).

30. The composition of claim 29 wherein said conjugate is a conjugate of haemoglobin and a non-toxic molecule with formula C17H16O6.

31. The composition of claim 21 or 28 wherein the non-toxic non-toxic nucleic acid cross linking agent has the following structural formula: 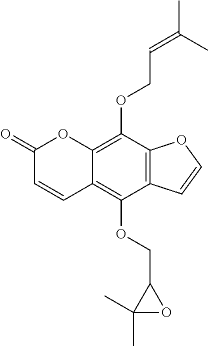 [SMILES: CC(C)=CCOc1c2occc2c(OCC2OC2(C)C)c2ccc(=O)oc12]
